(19) United States
(12) Patent Application Publication
(10) Pub. No.: US 2008/0261327 A1
(43) Pub. Date: Oct. 23, 2008

(54) Cell Free Assay for Determining a Substance of Interest and Molecular Complexes Used Therefore

(75) Inventor: Kevin Lee, New York, NY (US)

Correspondence Address: INVITROGEN CORPORATION;C/O INTELLEVATE, P.O. BOX 52050, MINNEAPOLIS, MN, 55402, US

(73) Assignee: Invitrogen Corporation, Carlsbad, CA (US)

(21) Appl. No.: 11/813,413

(22) PCT Filed: Jan. 4, 2006
(86) PCT No.: PCT/US06/00050

§ 371 Date: Feb. 13, 2008

Related U.S. Application Data
(60) Provisional application No. 60/641,867, filed on Jan. 5, 2005.

(51) Int. Cl. G01N 33/566; C07K 17/06; C12M 1/34

(52) U.S. Cl. 436/501; 530/402; 435/287.1

(57) ABSTRACT

The invention involves receptor complexes which include, inter alia, a receptor protein, and a reporter molecule. There is at least one unnatural, or non-naturally occurring amino acid in the receptor molecule. When a ligand interacts with the receptor, the interaction causes the reporter to generate a detectable signal. The complexes are useful in cell free, assay systems and may be used as part of micelles.

### FIELD OF THE INVENTION

This invention relates to cell free assays for determining if a substance of interest interacts with a molecule of interest, such as receptor protein. These assays are useful for identifying bioactive molecules, such as drugs, or for the production of biosensors or diagnostic devices. It also relates to the constructs which are useful in these cell free assays.

### BACKGROUND AND PRIOR ART

Cell based screening assays are tools well known to biologists. In these assays one investigates compounds of interest to determine, e.g., if the compounds modulate one or more biological process of interest.

One area where cell based screening assays have become widely accepted is the high-throughput analysis of materials for use as pharmaceuticals. In these assays the modulation of a given target by a compound of interest is coupled to a specific cellular readout. For example, the activation of a given cell surface receptor can elicit a change in the transcriptional profile of an enzymatic reporter gene, or the elevation of a given second messenger such as Ca²⁺, cAMP, or inositol triphosphate, which can be quantified, e.g., calorimetrically or fluorescently. Cell-based assays are useful and desirable because, unlike traditional binding assays, they have the potential to measure receptor modulating activity, a feature that is, ultimately, a requirement of drug functions. Cell-based screening assays also have several advantageous over animal model testing (e.g., lower expense, shorter assay period). High-throughput, cell-based screening assays can be scaled up via technologies such as “FLIPR,” “Leedseeker,” “VIPR,” and fluorescent, high speed cell-imaging.

Carrying out high-throughput, cell-based assays, however, presents a set of distinct challenges. Unlike biochemical reagents like enzymes, proteins, and membrane-bound receptors, cells are live, dynamic entities. In some cases the complex biological processes of a cell can negatively impact the proper expression and function of a recombinant target molecule in a heterologous cell system. There are numerous examples of target molecules (e.g., olfactory receptors) that can not be expressed in cell lines that are amenable to screening of target proteins that have a detrimental effect on the viability of the host cell when expressed at high levels. Cell-based assays are dependent upon the biological responsiveness of the cells in an appropriate assay platform, and the proper and consistent expression of the target molecule. Constructing a robust cell-based assay can be problematic for targets that have a negative effect on cell viability over time, as the cells which express the target molecule are subcultured. A further complication is the need to couple the activity of the target to a measurable cellular process, which can be very difficult in the case of uncharacterized targets (e.g., orphan receptors). Further, the miniaturization of cell-based screening assays is progressing, with smaller and smaller numbers of cells being used. As this occurs, sensitivity of the assay to variability increases rapidly and dramatically.

Due to the issues with cell-based screening assays, some but not all of which are discussed herein, there has been, and continues to be interest in cell free screening assays. Schmid, et al., Anal. Chem., 70:1331-1338 (1998), for example, discuss chip-based screening assays, as do Hovius, et al., Trends Pharmacol. Sci., 21:266-273 (2000), and Weiss, Nat. Struct. Biol., 7:724-729 (2000). These systems permit single molecule resolution studies, as are described by, e.g., Nie, et al., Annu. Rev. Biophys. Biomol. Struct., 26:567-596 (1997); Ambrose, et al., Chem. Rev., 99:2929-2956 (1999), Weiss, Science, 283:1676-1683 (1999). All references cited here and throughout this application are incorporated by reference in their entirety.

Besides numerous advantages in terms of lower set-up and reagent costs, and the ability to assay uncharacterized receptors, such chip-based assays offer the potential to assay multiple targets simultaneously. Multiplex, chip-based assays have the potential to profile the activity of a given compound on a battery of receptors to evaluate, for example, the selectivity of a given compound. These assays can also be used to identify a previously unknown receptor for a given ligand.

Such chip-based assays are not without problems, however. Basic to such assays is immobilization of the target molecule, such as a receptor protein, to the solid phase. Interactions between the solid phase and the immobilized molecule may lead to modification or loss of function of the immobilized molecule. Yet another issue which must be confronted is that these chip-based systems may not fairly replicate the environment in which many receptors function, including cell membrane bound receptors, such as the G-protein coupled receptors, or “GPCRs” as they will be referred to hereafter.

The GPCRs are integral membrane proteins. They constitute the largest family of receptors in the human genome, and are also seen in other mammalian, and non-mammalian species, including primates, canines, insects, and so forth. With respect to drug discovery, it has been estimated that over 50% of therapeutic agents that are on the market or are in development are directed at GPCRs as their target. See Edwards, et al., Trends Pharmacol. Sci., 21:304-308 (2000). Hence, there is considerable interest in developing cell free assays for target molecules in general, receptors in particular, and the GPCRs most particularly.

Neumann, et al., Chem. Bio. Chem., 3:993-998 (2002), discusses one form of cell free assay, using the human β2 adrenergic receptor (“β2AR.”) This is a GPCR that mediates the effect of catecholamines such as epinephrine released by the sympathetic nervous system. The paper referred to, supra discusses direct labeling of β2AR, using a chemical coupling reagent to attach a fluorophore to native cysteine residues Fluorophores coupled to an endogenous cysteine at position 265 in the β2AR sequence show a change in fluorescence intensity in response to an agonist-induced conformational change in the receptor protein. The β2AR receptor was also modified to include a “FLAG” sequence at the N-terminus to facilitate labeling with antibody, and a histidine tag of 6 histidines at the C-terminus. The fluorophore-labeled β2AR is immobilized on an avidin or streptavidin coated surface, using a biotinylated antibody that binds the N-terminal FLAG epitope. Jensen, et al., J. Biol. Chem., 276(12); 9279-9290 (2001), describes another system of this type, as does Bieri, et al., Nature Biotech., 17:1105 (1999):

While the approach disclosed by Neumann et al. and Jensen, et al. is not without interest, it requires the unique presence of a chemically reactive amino acid residue such as cysteine in a position that is sensitive to conformational changes in the receptor. Alternately, it requires the modification of each receptor sequence to introduce such a reactive amino acid residue in a conformation-sensitive position and to delete similarly reactive residues in other positions. It further requires that these modifications and the fluorophore coupling reaction do not impair the receptor activity and ligand binding properties. Because of these constraints, it would be useful to have an approach available to carry out cell free assays on any receptor where the native amino acid sequences of the receptor are modified as little as possible. Furthermore, it would be desirable if the site labeled with fluorophore is unique and can be positioned at will at any point within the receptor sequence.

The invention, which is set forth in the disclosure which follows, addresses these and other issues, as will be seen by consideration thereof

### BRIEF DESCRIPTION OF THE FIGURES

FIG. 1 shows how the invention functions.

FIG. 2 shows one application of the invention, where a receptor of interest is attached to a solid support.

FIG. 3 shows a further application of the invention, where a plurality of receptors, are presented on a microarray to determine which receptors are activated by a given test compound.

FIG. 4 presents a further application of the invention, which is a microarray of ordorant receptors (Ors) designed to determine if a particular compound or compounds are present in a test sample by monitoring the receptor complexes bound and activated by the compound.

### DETAILED DESCRIPTION OF PREFERRED EMBODIMENTS

In the description of the invention, reference will be made to both general, and specific embodiments of various portions thereof. Reference to specifics are given to explain the invention in greater detail, and/or to provide an example of particularly preferred embodiment. They are not to be taken as limiting the invention in any way.

In the practice of the invention, one requires a supply of receptor molecule of interest. While there are various, non-recombinant ways of obtaining pure, receptor proteins, it is most preferred, for reasons well known to the skilled artisan, to prepare these receptor molecules recombinantly. Such recombinant preparation may take place in a prokaryotic cell system, such as E. Coli, or a eukaryotic system, such as Spodoptera frugiperda, or some other cell type that is easily transformed or transfected with, e.g., a plasmid or viral expression vector.

In order to make the receptor molecules recombinantly, it is preferred to introduce a recombinant construct, e.g., an expression vector, into a host cell, such as those referred to supra.

The expression vectors used in the invention are constructed such that they comprise, at a minimum, a coding region which encodes a protein or a portion of a protein which facilitates movement of the receptor molecule of interest to the extracellular membrane, a coding region which encodes the receptor of interest, and a modification of the receptor, which facilitates binding of a reporter molecule thereto.

In a preferred embodiment of the invention, when E. coli cells are used as the host cells, the protein which facilitates the movement to the extracellular membrane is one which is normally transported to the periplasm of the cell. Maltose binding protein, or “MBP” is an example of one such protein. The construct encoding the MBP is placed 5′ to the construct which encodes the modified receptor. The construct is chosen such that a cleavage site is placed in between the two components, e.g., MBP and the receptor protein, so that at a convenient point in time, the transport protein may be cleaved therefrom. See Tucker, et al., Biochem. J., 317(Pt3):891-9 (1996), for a description of such an MBP system.

The receptor may be any of the receptors known to the art. The “G-protein coupled receptors” represent one family of great interest. Exemplary of the receptors in this family are the β2 adrenergic receptor, or “β2AR,” and serotonin receptors, such as 5HT1A, 5HT2C, and others that are well known to the art. Preferred systems for expression of GPCRs are well known. See, e.g., Tate, et al., Trends Biotechnol., 14(11):426-430 (1996).

The region encoding the receptor, as was noted supra, is modified such that, when translated, an unnatural non-naturally occurring, or chemically reactive amino acid residue is incorporated at a defined site within the receptor sequence. In other words, any amino acid other than the standard 20 amino acids described, e.g., in standard biochemistry textbooks, as being encoded by the genetic code. Numerous methods have been identified which allow the highly efficient incorporation of unnatural or modified amino acid using in vitro or in vivo translation systems. Examples of such systems include those described in: Cornish et al., Proc. Natl. Acad. Sci. USA, 91:2910-2914 (1994); Wang et al., Science, 292:498-500 (2001); Santoro et al., Nature Biotechnol., 20:1044-1048 (2002); Wang et al., Proc. Natl. Acad. Sci. USA, 100:56-61 (2003); Sakamoto et al., Nucl. Acids Res., 30:4692-4699 (2002); Zhang et al., Proc. Natl. Acad. Sci. USA, 101:8882-8887 (2004); Turcatti, et al., J. Biol. Chem., 271: 19991-19998 (1996), and Patent Application No. US 2003/0082575 A1. Using these methods, one may incorporate a unique fluorescent amino acid at a defined reporter position, or incorporate a unique chemically reactive amino acid, which may then be labeled with a fluorescent reporter molecule in a subsequent step. In an especially preferred embodiment of the invention, this is a ketone-containing amino acid, such as para-acetyl-L-phenylalanine described in Wang et al., Proc. Natl. Acad. Sci. USA, 100:56-61 (2003), which is subsequently labeled with a ketone-reactive fluorescent moiety, such as a fluorescent hydrazide derivative, or other commercially available ketone-reactive fluorophores.

The unnatural amino acid residue may be located at the C terminus of the receptor, or may be inserted at any point within the receptor which does not result in serious impairment of the receptor function. For example, in the case of the GPCRs described supra, the unnatural amino acid may be inserted in any of the cytoplasmic loops between the transmembrane domains, or in the cytoplasmic tail following the seventh transmembrane domain. As a further example, the GPCR may be expressed as a fusion to a G-protein, wherein the C-terminus of the receptor is fused to the N-terminus of the G-protein alpha subunit. See Milligan, Methods Enzymol., 343:260-273 (2002) for a discussion of such receptor-C protein fusion systems. In such fusions, the unnatural amino acid may be inserted at the junction between the receptor and the G-protein, or at the C-terminus of the G-protein fusion partner. More than one unnatural, or non-naturally occurring amino acid or other binding structure can be added to the molecule. Other specific sites for incorporation of the unnatural or non-naturally occurring amino acid are possible and these, as well as specific amino acids to incorporate as well as methods for their subsequent labeling, will be known to the skilled artisan.

Once the construct, referred to supra, is expressed, it is purified from the cell. Many methods are known for how to accomplish this, and as such will only be discussed briefly herein. As the constructs have been designed to transport the receptor protein of interest to the cell membrane, the purification protocol preferably isolates membrane fractions of the cells. “Proof of principle,” i.e., did the cells express the receptor of interest, can be determined very easily. Many receptors, including the GPCRs referred to supra, are known to contain an N-terminal region which crosses the cell membrane, followed by a plurality of domains which are transmembrane domains, and a C terminal, intracellular region. As this is the milieu in which these receptors function normally, one determines their presence and activity by adding a ligand known to bind to the receptor and then determining if binding occurs.

Once the receptor is known to be present in the membrane extract, it is solubilized by adding a detergent, preferably a non-ionic detergent, such as an alkyl maltoside, such as n-dodecyl-β-D maltoside. See Weiss, et al., “MRC Laboratory of Molecular Biology” available through NCBI, to show the use of this detergent, in purifying receptors. Other non-ionic detergents are known to the artisan, and need not be presented here.

The proteins are then separated via, e.g., affinity chromatography or other methods known in the art. The protein portion used to transport the receptor to the cell membrane may be removed at this point, but it need not be.

Labeling with a reporter molecule may be accomplished either in the native membrane of a cell or following isolation from the plasma membrane. Numerous methods for the purification of the receptor molecule exist and need not be reiterated here.

The reporter molecule may be any substance which generates a discernable signal upon interaction of the receptor with a ligand. The signal may be chemiluminescent, calorimetric, radioactive, and is preferably fluorescent.

In a preferred embodiment, the labeling of the receptor takes place in a solution of a non-ionic detergent, such as the detergent described supra, so as to form micelles which contain the complexes.

As noted, supra, in practice the complexes of the invention are used, in a cell free system such as a non-ionic detergent micelle, although this is not required. The complexes may also be incorporated into an artificial lipid bilayer. A substance of interest is then admixed with the indicator system, and changes in the fluorescence, such as fluorescence intensity, can be measured and compared to a control, to determine if there has been interaction, and if so, the extent thereof. Such a comparative assay can be used to determine agonistic or antagonistic properties of a molecule, such as by comparing signal obtained with the substance of interest to a value obtained using a known ligand.

The molecular complexes of the invention and the micelles which incorporate them may be used to produce apparatuses, where these materials are affixed to a solid phase, such as glass, plastic, or a microchip. Such apparatuses can contain multiple copies of one type of molecular complex or a mixture of various types of molecular complexes. Which type to use will depend upon the type of assay under construction.

Attachment to the solid phase may be accomplished in any of the ways known to the art. As was pointed out, supra, one embodiment of the invention includes maltose binding protein at the N-terminus. One can attach the molecular complexes to the solid phase, via an anti-MBP antibody, possibly via the intermediary of a biotin-(strept)avidin system. Other types of epitope tag, including but not being limited to MYC, HA, or FLAG could also be used, again possibly through the intermediary of a biotin-(strept)avidin system. Attachment may also be effected using a polyhistidine-metal chelation affinity system.

More details and the practice of the invention will be seen via a review of the example which follows.

### DETAILED DESCRIPTION OF THE PREFERRED EMBODIMENTS

### Example 1

This example describes the use of this invention to measure the ligand-mediated activation of a model receptor, the GPCR known as β2 adrenergic receptor, or “β2AR.” This example and the methodology described herein are applicable to any receptor, including but not being limited to, all GPCRs. In this example, a cysteine residue at position 265 in the third intracellular loop is substituted with a novel ketone-containing amino acid, para-acetyl-L-phenylalanine. Since the ketone functional group is absent in the side chains of the twenty common amino acids, and since this functional group is readily modified using a variety of different chemical strategies, the incorporation of this unnatural amino acid at a single position provides a convenient tag for site-selective modification of the receptor molecule with a fluorescent reporter moiety.

First, standard site-directed mutagenesis techniques are employed to convert the endogenous codon for Cys265 (TGC) in the human β2AR gene sequence to an amber stop codon (TAG). This position is chosen as an initial example since previous studies described supra have demonstrated that a fluorescent reporter attached to this site displays a measurable change in fluorescence properties in response to ligand-induced conformational dynamics of the β2AR. It is understood that other positions may be chosen instead, and the magnitude of the fluorescence change may be compared to select a most desired position for each receptor to be studied.

In this example, the receptor is further modified with a C-terminal tag such as a polyhistidine tag, FLAG tag, streptavidin binding peptide tag, or other tag for subsequent purification.

The resulting C terminally-tagged receptor gene is subcloned into a commercially available vector, i.e., PMAL-p2X, which already contains a coding region for maltose binding protein (MBP), in operable linkage. Further modifications of the MBP sequence are also possible to place one or more N-terminal epitope tags in the MBP sequence following the endogenous MBP signal sequence. Such tags may comprise a polyhistidine tag, FLAG tag, streptavidin binding peptide tag, or other tag for subsequent purification and study.

Next, the orthogonal tRNA-synthetase pair described in Wang et al., Proc. Natl. Acad. Sci. USA, 100:56-61, is employed to genetically incorporate the unnatural amino acid para-acetyl-L-phenylalanine with high fidelity in E. coli. This pair consists of a modified tyrosine amber suppressor tRNA (mutRNA^{Tyr}_{CUA}) and a modified Methanococcus jannaschii tyrosyl-tRNA synthetase that selectively and faithfully charges this tRNA molecule, and only this tRNA molecule, with the unnatural amino acid para-acetyl-L-phenylalanine. The genes encoding this tRNA-synthetase pair may be carried on the same plasmid as the receptor gene described supra or may be carried on a second compatible plasmid carrying a second antibiotic resistance gene.

The plasmid containing the MBP-receptor fusion gene is then transformed alone or together with the modified tRNA and synthetase genes into commercially available E. coli cells via standard methodologies. The strain of cells chosen will be obvious to the skilled artisan and will be selected for compatibility with the specific plasmids and promoters used to express the receptor, tRNA and synthetase genes. Such strains may include BL21, DH10B, or other readily available E. coli strains suitable for protein expression. Transformed cells are then plated onto LB plates containing appropriate antibiotics.

A single colony is selected, and used to inoculate culture medium containing appropriate antibiotics. The choice of culture medium and antibiotics will also be obvious to the skilled artisan and will be selected for compatibility with the bacterial strains and protein expression systems used. The culture is grown, at 37° C., to log-phase and stored overnight at 4° C. The culture is then centrifuged and resuspended in an equal volume of fresh medium containing appropriate antibiotics and then used to inoculate a fresh culture containing appropriate antibiotics at a dilution of 1:100. This culture is then grown to an OD₆₀₀ of approximately 0.5, at 37° C.

Temperature is reduced to 18° C., and IPTG is added to a 0.1 mM final concentration to induce expression of the receptor gene, and then cultures are grown for about 18 hours. For incorporation of the unnatural amino acid, the culture medium also includes 1 mM para-acetyl-L-phenylalanine. Cells are harvested, via centrifugation, and stored at −80° C.

Western analysis is performed, using a commercially available anti-MBP antibody or other antibodies directed to the chosen epitope tags, to confirm that expression of full-length receptor protein requires the presence of the modified tRNA-synthetase pair and the presence of para-acetyl-L-phenylalanine in the culture medium.

When needed, spheroplasts are prepared by resuspending cell pellets obtained from a 500 ml culture, in 60 mls of ice cold Tris (0.1M, pH8.0). An equal volume of ice-cold 0.1M Tris pH8.0/0.5M sucrose is added, and the cell suspension is mixed gently. EDTA is added, to a final concentration of 0.5 mM, as is lysozyme (0.05 mg/ml, final concentration), and DNAase I (10 ug/mil, final). The cell solution is incubated with rocking, for one hour at 4° C. Spheroplasts are then pelleted via centrifugation at 18,500×g for one hour, at 4° C.

Radiolabeled ligand binding studies using commercially available ³H-dihydroralprenolol (for the human β2AR) is performed using spheroplast prepared from bacteria expressing the modified tagged receptors to confirm the production of functionally active receptor capable of high affinity binding to an appropriate ligand and to establish that incorporation of the modified amino acid does not alter ligand binding properties.

For subsequent purification, the resulting spheroplast pellet is resuspended in 70 ml of ice cold Tris (pH 7.4, 20 nM) 20 mM NaCl and homogenized in a glass/glass homogenizer, using a tight fitting pestle.

The membranes are recovered via a centrifugation at 100,000×g for 2 hours, at 4° C. The membrane pellets are then resuspended in 10 mM of ice cold, 20 mM Tris (pH 7.4)/20 mM NaCl, and homogenized as above.

Membrane proteins are then solubilized, via addition of equal volume of 2% (w/v) n-dodecyl-β-D-1-maltoside/0.4% cholesteryl hemisuccinate. The solution is allowed to rock, overnight at 4° C., and remaining insoluble materials is pelleted via centrifugation at 12,000×g for one hour, and removed.

In this example, the ketone-containing receptor is first purified using a C-terminal streptavidin binding peptide (SBP) tag, labeled with a ketone-reactive fluorescent moiety, and then purified again using an N-terminal FLAG epitope tag. As is evident to the skilled artisan, other labeling and purification protocols may be chosen.

For the first purification step, Tris (pH 7.4) is added to the membrane proteins to a final concentration of 50 mM, NaCl is added to a final concentration of 300 mM, and avidin is added to a final concentration of 40 μg/ml. The solution also contains protease inhibitors (1 μM leupeptin, 1 μM pepstatin, and 1 μM PMSF). The solution is incubated with rocking at 4° C. for one hour, after which insoluble materials are removed via centrifugation, at 12,000×g for one hour, at 4° C. 1.5 ml of a 50% slurry of commercially available Streptavidin-agarose beads are added to the solubilized membranes, and are incubated, with rocking overnight, at 4° C.

The slurry is then loaded into a disposable column for chromatography, washed with 25 bed volumes of wash buffer 1 (50 mM Tris, pH 7.4, 300 mM NaCl, 0.2% maltoside/0.04% CHS), followed by a wash with 25 bed volumes of wash buffer 2 (50 mM Tris, pH 7.4, 300 mM NaCl, 0.05% maltoside/0.01% CHS). SBP tagged receptor protein is then eluted in elution buffer (50 mM Tris, pH 7.4, 300 mM NaCl, 2 mM desthiobiotin, 0.05% maltoside/0.01% CHS).

A commercially available ketone-reactive fluorescent labeling reagent is then added to direct selective labeling of the introduced para-acetyl-L-phenylalanine residue. As an example, for labeling with fluorescein hydrazide, receptor protein containing fractions are pooled, dialyzed against 100 mM Potassium phosphate, pH 6.5, 150 mM NaCl, 0.05% maltoside/0/01% CHS at 4° C. Fluorescein hydrazide is added to a final concentration of 1 mM and the reaction mixture is incubated for 18 hours at 25° C. Unreacted fluorophore is then removed by affinity purification using an N-terminal epitope tag such as a FLAG peptide or polyhistidine tag

For purification using an N-terminal FLAG tag, 0.5 ml of a 50% slurry of commercially available FLAG peptide resin is added to the labeled receptor protein, and are incubated, with rocking overnight, at 4° C.

The slurry is then loaded into a disposable column for chromatography, washed with 50 bed volumes of wash buffer 1 (50 mM Tris, pH 7.4, 150 mM NaCl, 0.05% maltoside/0.01% CHS, 1 mM CaCl2). Labeled receptor protein is then eluted in elution buffer (50 mM Tris, pH 7.4, 150 mM NaCl, 2 mM EDTA, 100 μg/ml FLAG peptide, 0.05% maltoside/0.01% CHS).

Western analysis is performed, using a commercially available anti-MBP antibody or other antibodies directed to the chosen epitope tags, to confirm that the preparation, labeling and isolation of the MBP-receptor fusion protein is successful. Radiolabeled ligand binding studies are performed to confirm that labeling does not alter ligand binding properties. Fluorescence spectroscopy is used to quantify the extent of the labeling reaction and to characterize the labeled protein.

Next, fluorescence spectroscopy of the reporter moiety is used to monitor ligand-induced changes in receptor conformation. In these experiments, the labeled receptor solution is monitored in a stirring cuvette while specific agonists and antagonists are added at various concentrations. The magnitude of the fluorescence change and response kinetics are evaluated. Preincubation with receptor antagonists and treatment with control compounds are used to verify that measured changes in fluorescence result from receptor conformational dynamics.

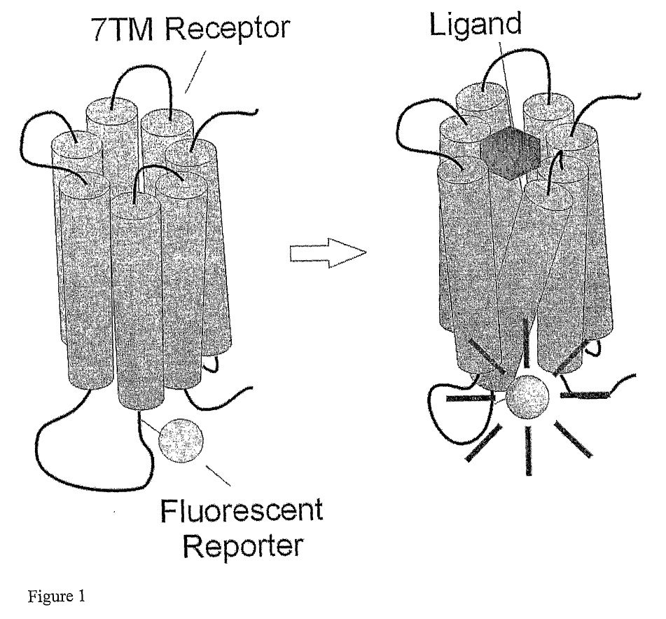

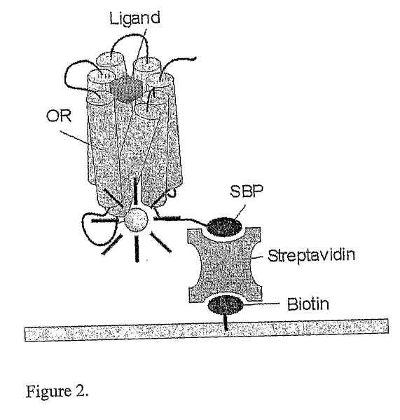

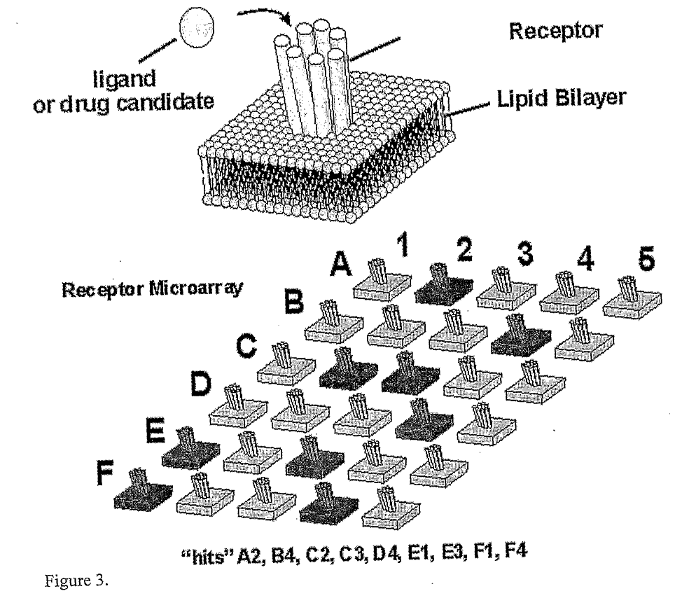

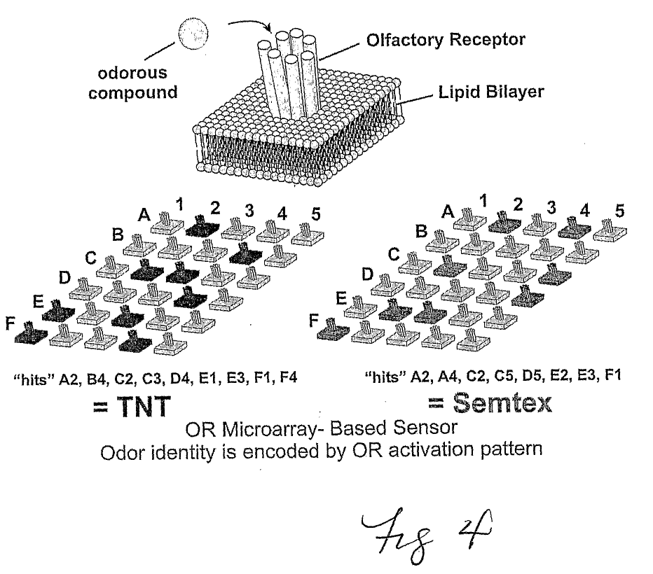

FIG. 1 shows how the invention is used to detect ligand-induced receptor changes. See, e.g., Sachmann, Science 271:43-48 (1996), incorporated by reference, for additional information on this system. The complexes can then be attached to solid phases, such as microchip arrays, as depicted in FIG. 2. In FIG. 3, a solid phase, such as a microarray, is presented, with a plurality of receptors bound thereto. Such a microarray may be used for determining which receptor or receptors are activated by a particular compound or compounds. These assays could be used for screening the activity of a given compound on a number of known receptors to determine, for example, the potential for undesired side-effects. Similarly, such a microarray assay can be used to identify receptors for a compound not known previously. A second embodiment of the invention can be seen in FIG. 4, where one can determine whether a particular compound or compounds are present in a sample, by screening with an array which includes one or more receptors that are activated by the compound(s).

Other aspects of the invention will be clear to the skilled artisan and need not be reiterated here.

We claim:

1. A substantially pure molecular complex which comprises:
(i) a receptor comprising a protein with an amino acid sequence, having an N terminus and a C terminus, said amino acid sequence containing at least one natural or non-naturally occurring amino acid, and
(ii) a reporter molecule linked to said receptor molecule via attachment to said unnatural or non-naturally occurring amino acid, wherein said reporter generates a detectable signal upon interaction of said receptor and a ligand which interacts with said receptor.

2. The substantially pure molecular complex of claim 1, wherein said reporter is covalently coupled to said unnatural or non-naturally occurring amino acid.

3. The substantially pure molecular complex of claim 1, wherein said reporter molecule is fluorescent.

4. The substantially pure molecular complex of claim 1, wherein said receptor is a GPCR.

5. The substantially pure molecular complex of claim 1, further comprising a protein or portion of a protein positioned at the N-terminus of the amino acid sequence of said receptor which facilitates transport of said molecular complex to an extracellular membrane of a cell.

6. The substantially pure molecular complex of claim 5, wherein said protein or portion of a protein is maltose binding protein or a portion thereof.

7. The substantially pure molecular complex of claim 1, wherein said unnatural or non-naturally occurring amino acid is positioned at the C terminus of said receptor.

8. The substantially pure molecular complex of claim 1, wherein said receptor comprises at least 6 transmembrane domains, and has an intracellular loop between the 5^{th} and 6^{th} transmembrane domains, and said unnatural or non-naturally occurring amino acid is positioned in said intracellular loop.

9. The substantially pure molecular complex of claim 1, wherein said receptor is fused to a G-protein alpha subunit to form a fusion protein and said unnatural or non-naturally occurring amino acid is positioned between said receptor and said G protein alpha subunit, or at the C-terminus of said fusion protein.

10. A micelle which comprises the substantially pure molecular complex of claim 1, and a non-ionic detergent.

11. The micelle of claim 10, wherein said non-ionic detergent is n-dodecyl-β-D-maltoside.

12. A method for determining if a substance interacts with a receptor, comprising contacting said substance to the substantially pure molecular complex of claim 1, and determining a detectable signal as an indication of interaction between said substance and said receptor.

13. A method for determining if a substance interacts with a receptor, comprising contacting said substance with the micelle of claim 10, and determining a detectable signal as an indication of interaction between said substance and said receptor.

14. A method for determining if a substance of interest is an antagonist of a receptor comprising contacting said substance to either the substantially pure molecular complex of claim 1 or the micelle of claim 10 in the presence of a known ligand for the receptor, detecting a signal, and comparing said signal to a signal obtained with said ligand alone, wherein a difference in said signals indicates that said substance is a possible antagonist for said receptor.

15. Apparatus useful in determining if a substance binds to a receptor, comprising the substantially pure molecular complex of claim 1 or the micelle of claim 10 affixed to a carrier.

16. The apparatus of claim 15, comprising a plurality of said substantially pure molecular complexes or micelles.

17. The apparatus of claim 16, wherein said plurality of substantially pure molecular complexes or micelles are the same.

18. The apparatus of claim 16, wherein said plurality of substantially pure molecular complexes or micelles are different.

19. The apparatus of claim 15, wherein said carrier is a glass slide, a plastic material, or a microchip.

20. A composition comprising a lipid bilayer having inserted therein the substantially pure molecular complex of claim 1.

21. A compound comprising a lipid bilayer having inserted therein a receptor comprising a protein with an amino acid sequence having an N-terminus and a C terminus, said amino acid sequence comprising at least one unnatural or non-naturally occurring amino acids.
